(19) United States
(12) Patent Application Publication
(10) Pub. No.: US 2005/0245809 A1
(43) Pub. Date: Nov. 3, 2005

(54) MAGNETIC RESONANCE IMAGING WITH IMPROVED DIFFERENTATION OF INFARCTED TISSUE

(75) Inventors: Steven Dana Wolff, New York, NY (US); Thomas Kwok-Fah Foo, Baltimore, MD (US)

Correspondence Address: ARTZ & ARTZ, P.C., 28333 TELEGRAPH RD., SUITE 250, SOUTHFIELD, MI, 48034, US

(73) Assignee: GE MEDICAL SYSTEMS GLOBAL TECHNOLOGY COMPANY, LLC, Waukesha, WI (US)

(21) Appl. No.: 10/709,365

(22) Filed: Apr. 29, 2004

(51) Int. Cl. A61B 5/05

(52) U.S. Cl. 600/410

(57) ABSTRACT

A method of generating a magnetic resonance image is provided, comprising subjecting a subject to a magnetic field. The subject comprised of a first tissue a second tissue and a third tissue. The method generates a first pulse sequence at a first TI time and generates a first image after the first pulse sequence. The first image has a first image first tissue magnitude, a first image second tissue magnitude, and a first image third tissue magnitude. The method then generates a second pulse sequence at a second TI time and generates a second image after the second pulse sequence. The second image has a second image first tissue magnitude, a second image second tissue magnitude, and a second image third tissue magnitude. Finally, the method generates a resultant image by combining the first image and the second image. The first image first tissue magnitude and the second image first tissue magnitude combine to form a positive resultant first tissue magnitude. The first image third tissue magnitude and the second image third tissue magnitude combine to form a negative resultant image third tissue magnitude.

### BACKGROUND OF INVENTION

The present invention relates generally to a magnetic resonance imaging method and assembly, and, more particularly to a magnetic resonance imaging method and assembly with improved differentiation of infarcted tissue.

Magnetic Resonance Imaging (MRI) is a well-known medical procedure for obtaining detailed, one, two and three-dimensional images of patients, using the methodology of nuclear magnetic resonance (NMR). MRI is well suited to the visualization of soft tissues and is primarily used for diagnosing disease pathologies and internal injuries.

Typical MRI systems include a super conducting magnet capable of producing a strong, homogenous magnetic field around a patient or portion of the patient; a radio frequency (RF) transmitter and receiver system, including transmitter and receiver coils, also surrounding or impinging upon a portion of the patient; a gradient coil system also surrounding a portion of the patient; and a computer processing/imaging system, receiving the signals from the receiver coil and processing the signals into interpretable data, such as visual images.

A variety of imaging methodologies are incorporated into magnetic resonance imaging technology. One such methodology is referred to as myocardial delayed enhancement (MDE). Myocardial delayed enhancement is a method by which infarcted tissue can be identified from normal myocardial tissue after a contrast media bolus has been delivered. Infarcted tissue retains a higher concentration of contrast media and appears to be bright or hyper-enhanced when visualized with a T₁-weighted imaging technique. Using this technique, tissues that have a delayed hyper-enhancement are considered non-viable tissue.

One known method of detecting the delayed hyper-enhancement phenomenon is to use a fast gradient-recalled imaging pulse sequence that is preceded by an inversion recovery preparation RF pulse. Contrast between the non-viable infarcted tissue and the normal myocardial tissue is obtained due to differences in the T₁ relaxation times. This technique is inefficient, however, since if the inversion time (TI time) does not null the normal myocardium tissue, the infarcted tissue contrast is reduced.

Various techniques have been proposed to obtain a more reliable method for suppressing the normal myocardial tissue. These techniques include the use of TI optimization at a plurality of TI times. The application requires a technologist to select the TI time that provides the optimal suppression of the myocardial tissue. This requires considerable time, effort, and technical skill in order to properly provide results. Another method utilized is the use of phase-sensitive reconstruction. Phase-sensitive reconstruction requires a separate (or additional) acquisition to calculate a phase correction to the images. Moreover, if the TI selected is greater than the null point of the normal myocardium, phase-sensitive IR reconstruction does not provide additional benefits over magnitude reconstruction. Additionally, as the T₁ relaxation times of infarcted tissue and blood are comparable, it can be difficult to differentiate the infarct-ventricular blood borders (i.e., the endo-cardial boundaries of the infarcted regions).

It would, however, be highly desirable to have a technique that provides an increased infarct-myocardial contrast over a wide range of TI times. Additionally, it would be highly desirable to have a technique that allowed for improved distinction between the ventricular blood and the endo-cardial borders of the infarcted tissue.

### SUMMARY OF INVENTION

A method of generating a magnetic resonance image is provided, comprising subjecting a subject to a magnetic field. The subject comprised of a first tissue a second tissue and a third tissue. The method generates a first pulse sequence at a first TI time and generates a first image after the first pulse sequence. The first image has a first image first tissue magnitude, a first image second tissue magnitude, and a first image third tissue magnitude. The method then generates a second pulse sequence at a second TI time and generates a second image after the second pulse sequence. The second image has a second image first tissue magnitude, a second image second tissue magnitude, and a second image third tissue magnitude. Finally, the method generates a resultant image by combining the first image and the second image. The first image first tissue magnitude and the second image first tissue magnitude combine to form a positive resultant first tissue magnitude. The first image third tissue magnitude and the second image third tissue magnitude combine to form a negative resultant image third tissue magnitude.

Other features of the present invention will become apparent when viewed in light of the detailed description of the preferred embodiment when taken in conjunction with the attached drawings and appended claims.

### BRIEF DESCRIPTION OF DRAWINGS

FIG. 1 a magnetic resonance imaging assembly in accordance with the present invention;

FIG. 2 is a graph of signal intensity curves at different inversion times for infarcted tissue, normal myocardium tissue, and blood;

FIG. 3 is an illustration of a proposed embodiment of the pulse sequence in accordance with the present invention;

FIG. 4 is an illustration of a series of TI images, the illustration showing the subtraction of first image from a second image to produce a resultant image; and

FIG. 5 is an illustration of signal difference curves using the present invention at three different reference TI times, the illustration showing the increased contrast in comparison to phase-sensitive reconstruction imaging.

### DETAILED DESCRIPTION

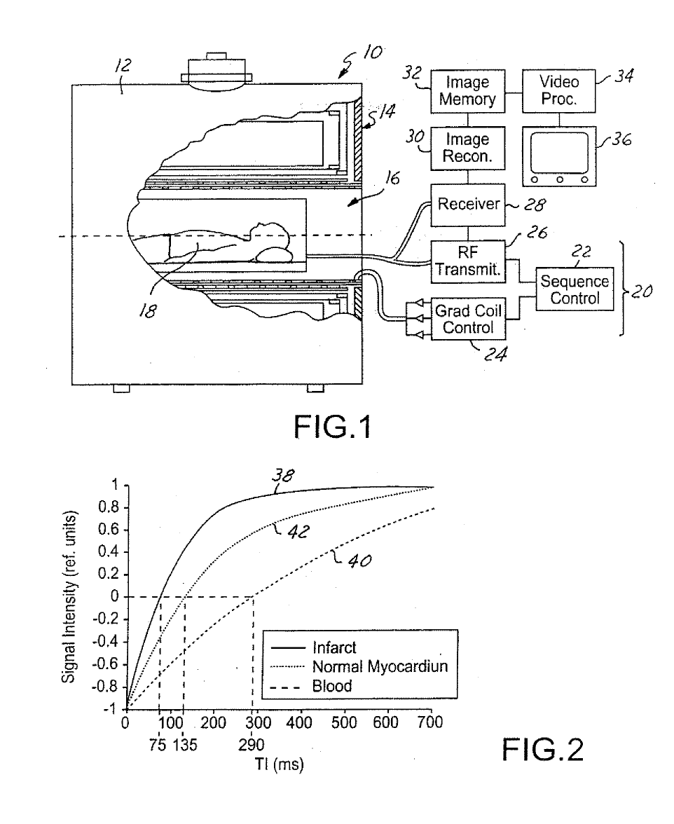

Referring now to FIG. 1, which is an illustration of a magnetic resonance assembly 10 in accordance with the present invention. Although a specific magnetic resonance assembly 10 is illustrated, it should be understood that the present invention is contemplated to be useful in a wide variety of magnetic resonance assemblies. The magnetic resonance imaging assembly 10 includes a superconducting magnet coil 12 positioned within a substantially cylindrical structure 14 defining a scanning bore 16 along a z-direction 17. An imaging object 18, such as a patient, is placed within the scanning bore 16. A series of coil controllers 20 in communication with a sequence controller 22 provide the assembly 10 with flexible control of the resonance assembly 10 functions. These coil controllers 20, such as a gradient coil controller 24 and a RF controller 26, produce changes in the magnetic field generated by the superconducting magnet coil 12. These changes are monitored by a receiver 28 which in turn is in communication with an image reconstructor 30. The image reconstructor 30 develops the data from the receiver 28 into a digital image that can be stored in image memory 32 and displayed on a video device 34. This allows the magnetic resonance assembly 10 to be utilized as a functional diagnostic device. A control station 36 allows a user to monitor and control operation of the magnetic resonance assembly 10.

The present invention utilizes the sequence controller 22 embedded with logic adapted to provide increased two tissue contrast (between a first tissue 38 and a third tissue 40) over a wide range of TI times from a multi-tissue subject (with a second tissue 42) see FIG. 2. In one specific embodiment, the present invention is directed towards providing increased contrast between infarcted tissue 38 and normal myocardial tissue 40 in a subject including blood 42. The present invention further improves distinction between the ventricular blood 42 and the endocardial borders of the infarcted tissue 38. This is accomplished through a unique sequence and imaging procedure. Although the present invention may be utilized to improve contrast between any three or more tissues, the present invention was directed towards differentiation between blood 42, myocardial tissue 40, and infarcted tissue 38.

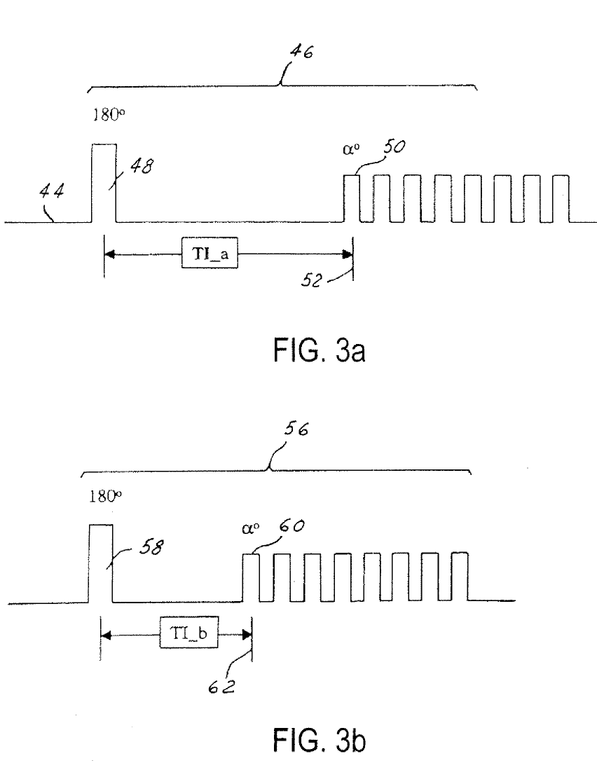

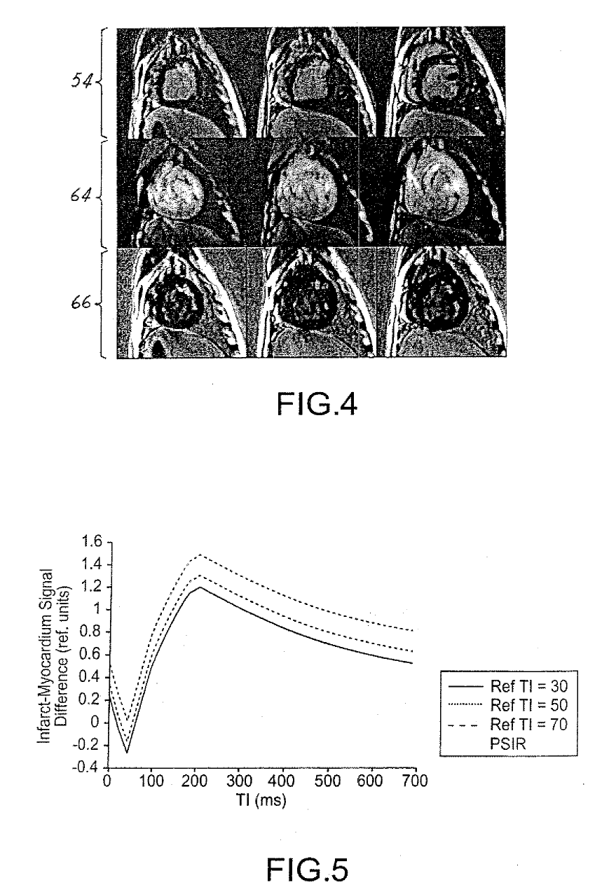

The present invention contemplates the introduction of a contrast media bolus into the subject. The introduction of contrast media is utilized to selectively modify the T₁ relaxation time of one of the tissues to be imaged. In the present example, the contrast media adjusts the T₁ of the infarcted tissue 38 to between 50-125 ms, depending on the time after introduction of the contrast. The present invention initially subjects the subject (patient) to a magnetic field 44. The invention then generates a first pulse sequence 46 (see FIG. 3). Although a variety of first pulse sequences 46 may be utilized, the present invention preferably utilizes a generated first inversion pulse 48 followed by a generated a first train of fast gradient-recalled echo rf pulses 50 at a first TI time 52. Note that in one embodiment, the acquisition is a segmented k-space acquisition of several k-space views acquired in each cardiac R-R interval. (The time between two consecutive heart beats.) Several R-R intervals are thus required to complete data acquisition. This first TI time is preferably a short TI time approximately coincident with the T₁ null time of the first tissue. Immediately after the first pulse sequence 46 is generated, a first image is taken 54 (see FIG. 4). This short TI time image is taken at or near the null point of the infarcted tissue 38. Although a variety of TI time values may be utilized, one embodiment contemplates the first TI time 52 being less than or equal to 100 ms. Since the concentration of the contrast media in the infarcted tissue 38 is higher than that in the ventricular blood 42 or normal myocardium 40, the longitudinal magnetization of the infarcted tissue 38 will have recovered above the Mz=0 line quickly. By imaging at a short TI time, the signal from the infarcted tissue 38 is small. The signal from the ventricular blood 42 will have higher signal intensity and the normal myocardium 40 will have the highest signal intensity due to the longer T₁ time.

The present invention further contemplates the use of a generated second pulse sequence 56. Again, although the second pulse sequence 56 can be varied, the present invention prefers the usage of a generated second inversion pulse 58 followed by a generated a second train of several gradient-recalled echo rf pulses (as part of a segmented k-space acquisition) 60 at a second TI time 62. This second TI time 62 is preferably a long TI time approximately coincident with the T1null time of the third tissue 40 (normal myocardium). Immediately after the second pulse sequence 56 is generated, a second image is taken 64 (see FIG. 4). This long TI time image is taken at or near the null point of the normal myocardial tissue 40. Although a variety of TI time values may be utilized, one embodiment contemplates the second TI time 62 being less than or greater to 200 ms. By imaging at a longer TI time, the signal from the normal myocardium 40 is small. The signal from the ventricular blood 42 will be higher, and the infarcted tissue 38 will have the highest signal intensity.

If TI_a is the first TI time. At this first inversion time 48, the signal intensities can be represented as S_infarct(a), S_blood(a), and S_myo(a), where S_infarct(a)<S_blood(a)<S_myo(a). When the second image 64 is acquired at the second TI time 62 of TI_b, where TI_b is preferably the null point of the myocardium 40, then the signal intensities can be represented as S_infarct(b), S_blood(b), and S_myo(b). At this second image time, the S_infarct(b) =S_blood(b) >>S_myo(b). FIG. 2 illustrates signal intensity curves for different inversion times for infarcted tissue 38 (T₁=75 ms), blood 42 (T₁=135 ms), and normal myocardium 40 (T₁=290 ms). It should be understood that these curves represent the post-contrast state where there is a detectable concentration of contrast media.

By taking the magnitude of the signal intensities of the different images and subtract the images at TI_b from that at TI_a, the resulting differences are:
Diff_infarct=|S_infarct(b)|−|S_infarct(a)|=S_infarct(b)
Diff_blood=|S_blood(b)|−|S_blood(a)|=small FIG.
Diff_myo=|S_myo(b)|−|S_myo(a)|<<0

The present invention using these two images generates a resultant image 66 by combining the first image 54 and second image 64 (see FIG. 4). Although the aforementioned example was accomplished through subtraction of absolute intensity values, other methods of combination are contemplated by the present invention. As it can be clearly seen, by subtracting the images acquired at the two different TI times, greater infarct-myocardial tissue contrast in addition to greater infarct-blood contrast is obtained. FIG. 5 illustrates the proposed subtraction approach using three different TI times. The null point for the infarcted tissue 38 in this example is 52ms. The signal difference curves are generated at different TI times, using TI_a=30,50, and 70 respectively. These curves demonstrate how the signal differences vary as a function of the TI_a times used. The optimum signal differences are obtained when TI_a is approximately that of the null point of the first tissue (infarct) 38.

The present invention allows the user to select a combined acquisition at a long and short TI time, without having to “tune” the TI time to suppress normal myocardial tissue 40. The subtraction technique also allows much greater contrast to be generated than can be realized with most conventional imaging techniques. It should be understood that the proposed invention can be combined into a single acquisition where two different images, each at a long and short TI time, are generated. This can be done to remove the possibility of mis-registration between the two different acquisitions. By obviating the need for careful selection of the TI time needed to suppress normal myocardial tissue 40 in cardiac viability studies, the present invention improves the speed, accuracy and reliability of imaging in these studies.

While particular embodiments of the invention have been shown and described, numerous variations and alternative embodiments will occur to those skilled in the art. Accordingly, it is intended that the invention be limited only in terms of the appended claims.

1. A method of generating a magnetic resonance image comprising:
subjecting a subject to a magnetic field, said subject comprised of a first tissue a second tissue and a third tissue;
generating a first inversion radiofrequency pulse;
generating a first train of radiofrequency pulses a first TI time
taking a first image after said first train of radiofrequency pulses;
generating a second inversion pulse;
generating a second train of radiofrequency pulses at a second TI time, said second TI time greater than said first TI time;
taking a second image after said second train of radiofrequency pulses; and
generating a resultant image by combining said first image and said second image.

2. A method as described in claim 1, wherein said first TI time is approximately coincident with a T₁ null time of the first tissue and wherein said first train of radiofrequency pulses constitute a data acquisition segment in a segmented K-space acquisition.

3. A method as described in claim 1, wherein said second TI time is approximately coincident with a T₁ null time of the third tissue.

4. A method as described in claim 1, wherein said first TI time is less than 100 ms.

5. A method as described in claim 1, wherein said second TI time is between 150 and 250 ms.

6. A method as described in claim 1, further comprising:
introducing a contrast agent prior to taking said first image.

7. A method as described in claim 1, wherein:
said first tissue comprises infarcted tissue;
said second tissue comprises blood;
said third tissue comprises normal myocardium tissue;
said first image nullifies said infarcted tissue;
said second image nullifies said normal myocardium tissue; and
said resultant image shows improved differentiation between said infarcted tissue and said normal myocardium tissue.

8. A method as described in claim 1, wherein said second inversion pulse is generated immediately after taking said first image.

9. A method as described in claim 1, wherein:
said first tissue comprises a first T₁ relaxation time;
said second tissue comprises a second T₁ relaxation time;
said third tissue comprises a third T₁ relaxation time;
wherein said third T₁ relaxation time is greater than said second relaxation T₁ time and said third T₁ relaxation time is greater than said first T₁ relaxation time.

10. A method as described in claim 9, wherein said second T₁ relaxation time is greater than said first T₁ relaxation time.

11. A method as described in claim 6, wherein the concentration of said contrast agent is higher in said first tissue than in either said second tissue or said third tissue.

12. A method as described in claim 1, wherein said combining said first image and said second image comprises subtracting said first image from said second image.

13. A method of generating a magnetic resonance image comprising:
subjecting a subject to a magnetic field, said subject comprised of a first tissue a second tissue and a third tissue;
generating a first pulse sequence at a first TI time;
generating a first image after said first pulse sequence, said first image having a first image first tissue magnitude, a first image second tissue magnitude, and a first image third tissue magnitude;
generating a second pulse sequence at a second TI time;
generating a second image after said second pulse sequence, said second image having a second image first tissue magnitude, a second image second tissue magnitude, and a second image third tissue magnitude; and
generating a resultant image by combining said first image and said second image, said first image first tissue magnitude and said second image first tissue magnitude combining to form a positive resultant first tissue magnitude, said first image third tissue magnitude and said second image third tissue magnitude combining to form a negative resultant image third tissue magnitude.

14. A method as described in claim 13, wherein said first image second tissue magnitude and said second image second tissue magnitude combine such that said resultant image second tissue magnitude is less than said second image second tissue magnitude.

15. A method as described in claim 13, wherein:
said first TI time is approximately coincident with a T₁ null time of said first tissue; and
said second TI time is approximately coincident with a T₁ null time of said third tissue.

16. A method as described in claim 13, wherein:
said first tissue comprises infarcted tissue;
said second tissue comprises blood; and
said third tissue comprises normal myocardium tissue.

17. A method as described in claim 13, further comprising:
introducing a contrast agent into said subject prior to generating said first image.

18. A magnetic resonance imaging assembly for imaging a subject comprised of a first tissue, a second tissue, and a third tissue, the magnetic resonance imaging assembly comprising:
a controller comprising logic adapted to:
generate a first inversion pulse;
generate a first train of gradient-recalled echo radiofrequency pulses at a first TI time;
generate a first image after said first train of gradient-recalled echo radiofrequency pulses;
generate a second inversion pulse;
generate a second train of gradient-recalled echo radiofrequency pulses at a second TI time, said second TI time greater than said first TI time;
generate a second image after said second train of gradient-recalled echo radiofrequency pulses; and
generate a resultant image by digitally combining said first image and said second image.

19. A magnetic resonance imaging assembly as described in claim 18, wherein:
said first TI time is approximately coincident with a T₁ null time of the first tissue; and
said second TI time is approximately coincident with a T₁ null time of the third tissue.

20. A magnetic resonance imaging assembly as described in claim 18, wherein said logic is further adapted to:
generate said resultant image by subtracting said first image from said second image.

21. A magnetic resonance imaging assembly as described in claim 18 wherein the said logic comprises an image acquisition segment segmented in k-space and ECG-gated to freeze cardiac motion.

22. A magnetic resonance imaging assembly as described in claim 18 wherein the said logic comprises an image acquisition segment comprising a train of gradient-recalled echo rf pulses.

23. A magnetic resonance imaging assembly as described in claim 18 wherein the said logic comprises an image acquisition segment comprising either gradient recalled echoes or steady-state free-precession sequences.
